(19) United States
(12) Patent Application Publication
(10) Pub. No.: US 2008/0275427 A1
(43) Pub. Date: Nov. 6, 2008

(54) Threaded catheter connector, system, and method

(76) Inventor: Shahn S. Sage, Andover, MN (US)

Correspondence Address: MUETING, RAASCH & GEBHARDT, P.A., P.O. BOX 581336, MINNEAPOLIS, MN, 55458-1336, US

(21) Appl. No.: 11/799,165

(22) Filed: May 1, 2007

(51) Int. Cl. A61M 25/16; A61M 31/00

(52) U.S. Cl. 604/533; 604/513

(57) ABSTRACT

Catheter connectors, connection systems, and methods in which a catheter is attached to a threaded connector that is inserted into the lumen of the catheter. The catheter may include an elastically compressible inner body that is surrounded by a reinforcing braid that is further surrounded by an optional outer jacket. When the connector is inserted into the lumen of the catheter, the elastically compressible inner body of the lumen may be compressed by and may conform to the shape of the threaded outer surface of the connector to form a fluid-tight seal between the lumen and the connector.

### TECHNICAL FIELD

The present invention relates generally to a medical connection system and, more particularly, to threaded catheter connectors, connection systems, and methods.

### BACKGROUND

In many medical applications, it is necessary to connect one section of medical tubing, e.g., a catheter, with another. Generally speaking, it is important that these connections be relatively secure and stable so that the catheter does not separate or develop leaks at the connection point. Security and leak-resistance take on special importance in applications where the catheter sections are implanted in a human body.

One procedure that necessitates implantation of a catheter into the body involves the use of an implantable medical device, e.g., a drug infusion pump. Such implantable medical devices are often used to control pain and/or spasticity, as well as to provide one or more drugs or fluid medications to a particular location within the body. For instance, a typical implant procedure may involve implanting a drug infusion pump into a cavity or subcutaneous pocket in the body and delivering a drug, via catheter(s), to an epidural space or intrathecal space of the spinal column or to a particular location within the brain.

A distal catheter section may be positioned in the desired location in the body and then connected to a proximal catheter section by use of a connector. The connection may be made by inserting one end or prong of the connector into a lumen of one catheter section (e.g., the proximal section) and the other end of the connector into the lumen of the other catheter section (e.g., the distal section) and then sliding both catheter sections towards one another (toward the middle of the connector). The proximal section may then be connected to the drug infusion pump.

While adequate, difficulties have been encountered in the manufacture and use of such prior art connectors. For example, an inadequate seal between the catheter and the connector may be formed during assembly or the catheters and/or connector may be damaged during assembly. Also, these connectors, which have been sized to fit within the lumens of the catheter sections, are small and may be difficult to manipulate during implantation. Moreover, because some of these connectors fit entirely within the lumens of the respective catheter sections, it is often difficult for the implanting clinician (e.g., a surgeon) to be sure that the interface between catheter sections is positioned at, or even near, the center of the connector (i.e., it may be difficult to center the catheter sections on the connector). Misalignment of the connector can result in a weakened connection that is more likely to separate and/or develop leaks. Other potential problems include a lack of ability to adequately secure the catheters relative to one another and an inability to provide sufficient strain relief to the connection.

### SUMMARY OF THE INVENTION

The present invention provides catheter connectors, connections systems, and methods in which a catheter is attached to a threaded connector that is inserted into the lumen of the catheter. The catheter may include an elastically compressible inner body that is surrounded by a reinforcing braid that is further surrounded by an optional outer jacket. When the connector is inserted into the lumen of the catheter, the elastically compressible inner body of the lumen may be compressed by and may conform to the shape of the threaded outer surface of the connector to form a fluid-tight seal between the lumen and the connector.

In one aspect, the present invention provides a catheter connection system that includes a connector body having an intermediate section and a first connector extending from the intermediate section, wherein a bore extends through the first connector and the intermediate section, and wherein the first connector has a threaded outer surface. The system also includes a catheter having an end portion attached to the first connector, wherein the end portion has a lumen that extends through the end portion towards a distal end of the catheter, wherein the lumen is located within an elastically compressible inner body that is surrounded by a reinforcing braid located around an outer surface of the inner body. When the first connector is located within the lumen in the end portion of the catheter, the elastically compressible inner body is compressed by and conforms to the shape of the threaded outer surface of the first connector to make a fluid-tight seal between the bore in the connector body and the lumen of the catheter.

In various aspects, the catheter connection systems of the present invention may optionally include one or more of the following features: when the first connector is located within the lumen in the end portion of the catheter, the outer dimensions of the catheter within the end portion occupied by the first connector remain substantially unchanged as compared to the outer dimensions of the catheter when the first connector is not located within the lumen; the inner body in the end portion of the catheter may have a wall thickness that is greater than a thread depth of the threaded outer surface of the first connector; the inner body in the end portion of the catheter may have a wall thickness that is about 25% or more of a diameter of the lumen before the first connector is located therein; the intermediate section of the connector body may include a pair of wrench flats located on an outer surface of the intermediate section, wherein the wrench flats are located on opposite sides of a longitudinal axis extending along the bore; the reinforcing braid may include at least one inelastic strand wound around the outer surface of the inner body; the reinforcing braid may include at least one non-metallic inelastic strand wound around the outer surface of the inner body; the inner body may include silicone; the inner body may consist essentially of silicone; the reinforcing braid and the inner body of the catheter may be surrounded by an outer jacket covering the outer surface of the inner body and the reinforcing braid.

The catheter connection systems of the present invention may also include a second connector extending from the intermediate section of the connector body, wherein the bore extending through the first connector and the intermediate body also extends through the second connector; and a proximal catheter having an end portion attached to the second connector, wherein the end portion of the proximal catheter has a lumen in fluid communication with the bore. The second connector may have a threaded outer surface and the end portion of the proximal catheter may have an elastically compressible inner body defining the lumen in the proximal catheter, the inner body being surrounded by a reinforcing braid located around an outer surface of the inner body. In such a system, when the second connector is located within the lumen in the end portion of the proximal catheter, the elastically compressible inner body may be compressed by and conform to the shape of the threaded outer surface of the second connector to make a fluid-tight seal between the bore in the connector body and the lumen of the proximal catheter. The second connector in such systems may include any of the features described in connection with the first connector.

In another aspect, the present invention provides a method of connecting a catheter to a connector, the method including providing a connector body that includes an intermediate section and a first connector extending from the intermediate section, wherein a bore extends through the first connector and the intermediate section, and wherein the first connector has a threaded outer surface; and inserting the first connector in a lumen at an end of a catheter such that the first connector occupies an end portion of the catheter, wherein the lumen is located within an elastically compressible inner body that is surrounded by a reinforcing braid located around an outer surface of the inner body of the catheter. The inserting includes rotating the first connector and the catheter relative to each other about an axis extending through the first connector and the lumen, wherein the inner body of the catheter is compressed by and conforms to the threaded outer surface of the first connector to make a fluid-tight seal between the bore in the connector body and the lumen of the catheter.

In yet another aspect, the present invention provides a therapeutic substance delivery system that includes an implantable therapeutic substance delivery device; a delivery catheter; and a catheter connector connecting the delivery catheter to the implantable therapeutic substance device. The catheter connector has a connector body that includes an intermediate section and a first connector extending from the intermediate section, wherein a bore extends through the first connector and the intermediate section, and wherein the first connector has a threaded outer surface. An end portion of the delivery catheter is occupied by the first connector, wherein the end portion has a lumen that extends through the end portion towards a distal end of the delivery catheter, and wherein the lumen is located within an elastically compressible inner body that is surrounded by a reinforcing braid located around an outer surface of the inner body. The elastically compressible inner body in the end portion of the delivery catheter is compressed by and conforms to the shape of the threaded outer surface of the first connector to make a fluid-tight seal between the bore in the connector body and the lumen of the delivery catheter.

The above summary is not intended to describe each embodiment or every implementation of the present invention. Rather, a more complete understanding of the invention will become apparent and appreciated by reference to the following Detailed Description of Illustrative Embodiments and claims in view of the accompanying figures of the drawing.

### BRIEF DESCRIPTION OF THE VIEWS OF THE DRAWING

The present invention will be further described with reference to the views of the drawing, wherein:

FIG. 1 is a perspective view of one illustrative connector body that may be used in a catheter connector system according to the present invention.

FIG. 2 is a side view of the connector body of FIG. 1.

FIG. 3 is a top view of the connector body of FIG. 1.

FIG. 3A is a partial cross-sectional view of the first connector of FIG. 1 taken along the bore.

FIG. 4 is an end view of the catheter connector body of FIG. 1.

FIG. 5 is a perspective view of one illustrative catheter body construction that may be used with the catheter connection system of the present invention (with layers partially removed).

FIG. 6 is a cross-sectional view of FIG. 5 taken along the bore depicted in FIG. 5.

FIG. 7 is a perspective view of the connector body of FIG. 1 attached to a delivery catheter.

FIG. 8 depicts one illustrative embodiment of an implantable medical device, a delivery catheter, and a threaded catheter connector.

FIG. 9 depicts another illustrative embodiment of an implantable medical device and delivery catheter connected thereto using a proximal catheter and a threaded catheter connector.

### DETAILED DESCRIPTION OF ILLUSTRATIVE EMBODIMENTS

In the following detailed description of illustrative embodiments of the invention, reference is made to the accompanying figures of the drawing which form a part hereof, and in which are shown, by way of illustration, specific embodiments in which the invention may be practiced. It is to be understood that other embodiments may be utilized and structural changes may be made without departing from the scope of the present invention.

As used herein, “a,” “an,” “the,” “at least one,” and “one or more” are used interchangeably. The term “and/or” (if used) means one or all of the listed elements or a combination of any two or more of the listed elements.

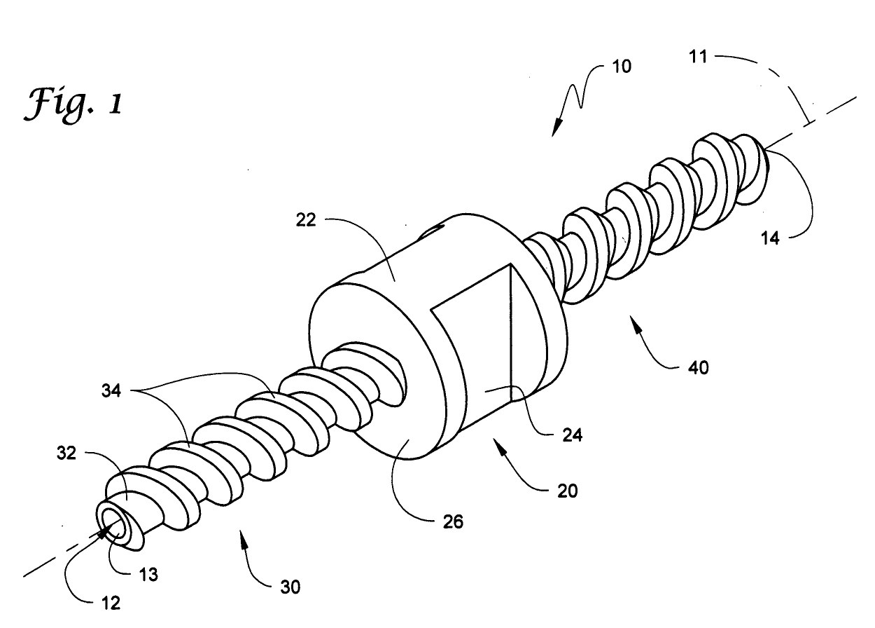

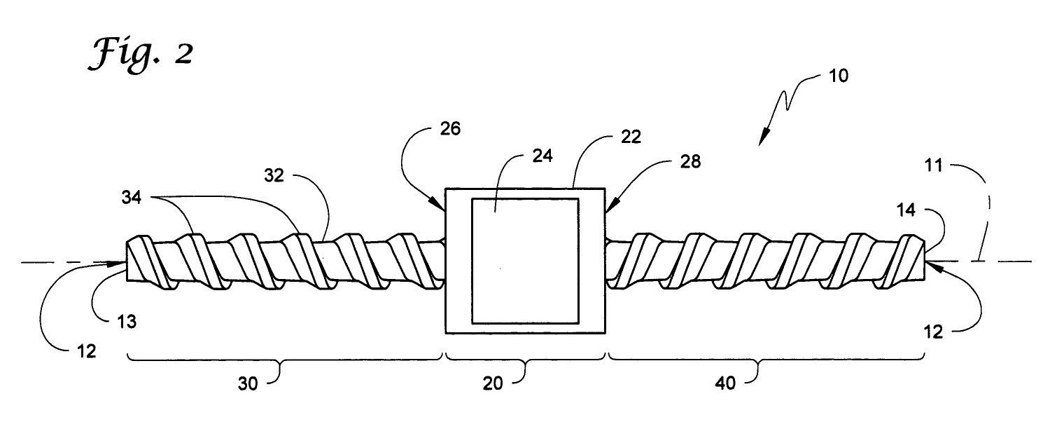

FIGS. 1-4 are different views of one illustrative connector body 10 used to connect catheters according to the present invention. The connector body 10 depicted in FIGS. 1-4 includes an intermediate section 20 that is located between a first connector 30 and a second connector 40, with the components being arranged along a longitudinal axis 11.

Although the connector body 10 depicted in FIGS. 1-4 is a two-sided threaded connector, i.e., it includes threaded connector structures on each of two ends, connector bodies of the invention may include only one side that includes a threaded connector as described herein. A single-sided connector body may, for example, be formed as integral component of, e.g., a pump or other device to which a catheter is to be connected.

In other embodiments, the connector bodies of the present invention may be two-sided, but include different connector structures on the two ends. For example, one side of the connector body may include a threaded connector (examples of which are described herein), while the other side of the connector body may include a different connector structure. Examples of some potentially suitable connector structures may be described in, e.g., U.S. Pat. Nos. 4,929,236 (Sampson); 4,963,133 (Whipple); 5,129,891 (Young); 5,637,102 (Tolkoff et al.); 5,405,339 (Kohnen et al.); and 6,910,906 (Schorn). Still other potentially suitable connector structures may be described in U.S. Patent Application Publication Nos. US 2005/0253389 (Schulte) and US 2006/0195066 (Cross, Jr.).

Furthermore, the connector bodies on which threaded connector structures may be provided may include more than two connector structures. For example, the connector bodies may include three or more connector structures, one or more of which may be threaded connector structures as described herein. One example of a connector body that includes three connector structures is described in, e.g., U.S. Patent Application Publication No. US 2005/0245887 (Olsen et al.).

In the depicted connector body 10, a bore 12 extends through the connector body 10 (along the longitudinal axis 11) such that fluids can pass through the connector body 10 from or to an attached catheter. The first connector 30 includes a first opening 13 that is in fluid communication with the bore 12. The bore 12 extends from the opening 13, through the first connector 30, through the intermediate section 20, through the second connector 40, and to the second opening 14. In other words, the first opening 13 is in fluid communication with the second opening 14 through the bore 12. The bore 12 is defined by the interior surface 15 in the connector body 10 and is, in the depicted embodiment, centered along the longitudinal axis 11 of the connector body 10. The bore 12 may, in some embodiments, not necessarily be centered along the longitudinal axis 11 of the connector body 10.

In the following description, the features of the threaded connector 30 will be described with the understanding that the description of the features of connector 30 apply as well to the features of the second threaded connector 40 (but will not be repeated in the interest of brevity). Although the first and second connectors 30 and 40 are depicted as the same (i.e., having the same thread pitch, size, length, etc.), the connectors 30 and 40 may be different in any of one or more respects.

The first connector 30 depicted in FIGS. 1-4 includes a thread 34 forming a threaded outer surface on the exterior 32 of the first connector 30. The thread 34 may take any suitable form or shape. Further, the thread pitch (spacing between successive threads) may vary depending on the materials used, the catheter to be connected to the connector 30, etc. For example, the thread pitch may range from, e.g., 2 threads per millimeter (mm) to about 5 threads per mm. Although the pitch of the thread 34 on connector 30 is depicted as constant along the length of the connector 30, it may alternatively change along the length of the connector 30.

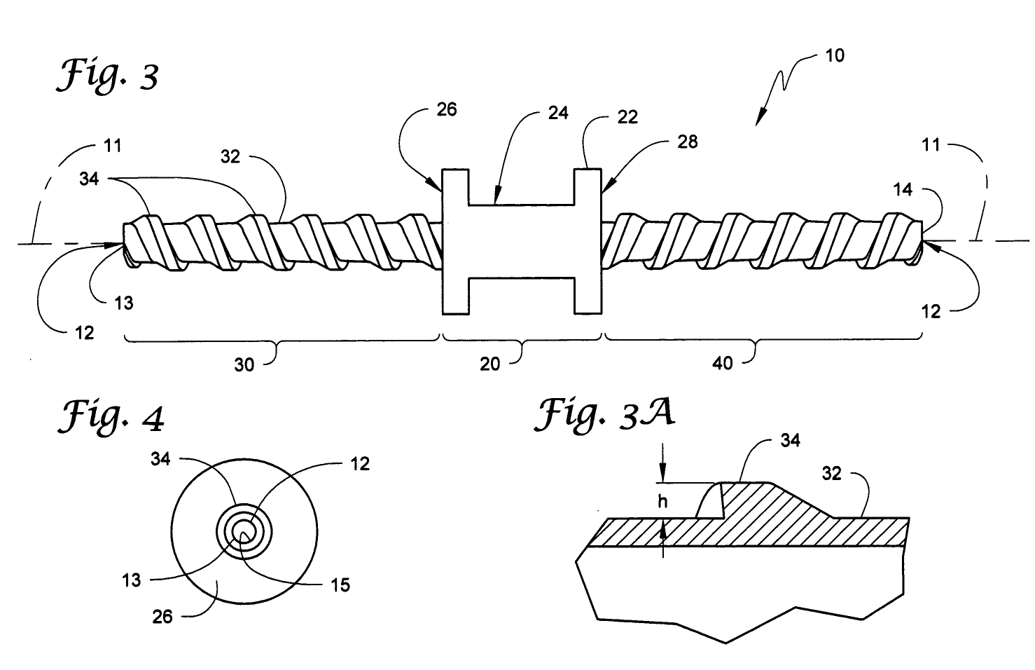

As depicted in FIG. 3A, the thread 34 may be characterized as having a thread depth, d, that is the distance from the outer surface of the wall 32 (which defines the innermost cylinder from which the thread 34 protrudes to the outermost portion of the thread 34).

The distal tip (proximate the opening 13 of the bore 12) of the connector 30 depicted as a part of the connector body 10 in FIGS. 1-4 may be cylindrically shaped which may conform to the shape of the lumen of a catheter into which the connector 30 will be inserted. Although not shown, the distal tip of the connector 30 may be tapered, conical, or take any other suitable shape to assist with insertion of the connector 30 into the lumen of the catheter.

The connector 30 may have any suitable outer cross-sectional shape (e.g., circular, oval, rectangular, octagonal, elliptical, etc.) that facilitates its insertion into differently-shaped lumens. Although the outer cross-sectional shape and the cross-sectional shape of the bore 12 formed through the connector body 10 may be the same (e.g., both the outer surface of the connectors 30 and 40 and the bore 12 may be circular), they may also be different (e.g., the outer surface of the connector 30 may be octagonal and the bore 12 may be circular, etc.).

As used herein, the term “diameter” may refer to effective diameter (greatest cross-sectional dimension taken perpendicular to a longitudinal axis) of any component, whether it has a circular or non-circular cross-sectional shape. The first connector 30 may, for example, have an outer diameter of about 0.25 mm to about 0.64 mm and a length of about 2.5 mm to about 5.0 mm. However, the connector body 10, or the components thereof, may have any suitable diameters and/or lengths depending on the application (i.e., connectors constructed with dimensions outside of these exemplary ranges may still fall within the scope of the present invention).

The intermediate section 20 of the depicted connector body 10 includes a generally cylindrical outer surface 22, wrench flats 24, and stop surfaces 26 and 28. Wrench flats 34 may be located on opposite sides of the longitudinal axis 11 that extends through the connector body 10. Although the intermediate section 20 is generally cylindrical in shape, the intermediate section 20 may take any suitable alternative shape, e.g., spherical, rectangular, etc.

The wrench flats 34 may provide surfaces on which a wrench (or any other suitable tool) may operate to facilitate relative rotational movement between the connector body 10 and a catheter to be connected to the connector body 10.

The relative rotational movement may essentially allow a user to turn or screw the threaded connector 30 into catheter lumens as described herein.

The stop surfaces 26 and 28 on the intermediate section 20 of the connector body 10 depicted in FIGS. 1-4 are generally perpendicular to the axis 11 along which the connectors 30 and 40 extend. The stop surfaces 26 and 28 may, however, take any other suitable shape or orientation. The stop surfaces 26 and 28 may provide a positive stop against which the ends of the catheters abut when attaching catheters to the connector body 10. Advancement of catheters on the connectors 30 and 40 past the stop surfaces 26 and 28 is difficult, if not impossible. By providing stop surfaces 26 and 28 that allow for both visual and tactile feedback, a clinician may ensure that each catheter is properly engaged with the connector body 10.

The various parts of the connector body 10 (the intermediate section 20 and the connectors 30 and 40) may be formed as a one-piece, completely integral body or, alternatively, the connector body may be assembled/constructed from two more different components. For example, the intermediate section 20 may be formed as one article with a hollow threaded shaft inserted therethrough (by, e.g., insert molding, etc.), where the connectors 30 and 40 are formed by the hollow threaded shaft. The connector body 10, or the components thereof, may be formed of any acceptable biocompatible material(s) including polymeric material, noble metals (e.g., titanium), stainless steel, etc.

Although not shown, the connection system may include a boot or sleeve that extends over the connector body 10 and the catheter(s) proximate the connector body 10. Such a boot or sleeve may provide a smoother, more biocompatible surface and may provide some strain relief for the connections.

Catheter bodies that may be useful in connection with connectors such as, e.g., the threaded connectors described herein may possess a number of features or characteristics. For example, catheters may be constructed such that they conform to the shape of the connector inserted into the proximal end of the lumen to form a fluid-tight seal between the outer surface of the connector and the inner surface of a lumen into which the connector is inserted. As used herein, a “fluid-tight seal” means a seal that prevents the passage of fluids delivered through the catheter when implanted into the body of a subject (it being understood that almost any seal can be compromised by the application of fluid pressure beyond the intended working limit of the seal). After, for example, a threaded connector (such as, e.g., connector 30) is inserted into a catheter lumen, the catheter may generally conform to the shape of the threaded connector to form a fluid-tight seal between the catheter and the connector.

Another potentially beneficial characteristic of catheters used in connection with the present invention is that they may preferably possess enhanced abilities to transmit torque along their length. For example, the connectors described herein use a threaded connector that may be introduced into the catheter lumen by rotation. That rotation will tend to twist a catheter into which the connector is being inserted (with the twisting being experienced as a torque exerted on the catheter about its longitudinal axis). It may be beneficial, therefore, if the catheter itself is relatively stiff such that it can, e.g., resist the torque exerted by the threaded connector during insertion. Catheters that are more resistant to torque forces may remain more stable during insertion of a threaded connector as compared to catheters that are less resistant to torque forces.

The reinforcing braid and optional outer jacket on catheters that may be beneficially used in connection with the present invention may also provide improvements in crush resistance, kinking, elongation, etc. These enhanced physical characteristics may be particularly helpful in catheters that include elastically compressible inner bodies that, themselves, have significantly limited ability to resist crushing, kinking, elongation, etc.

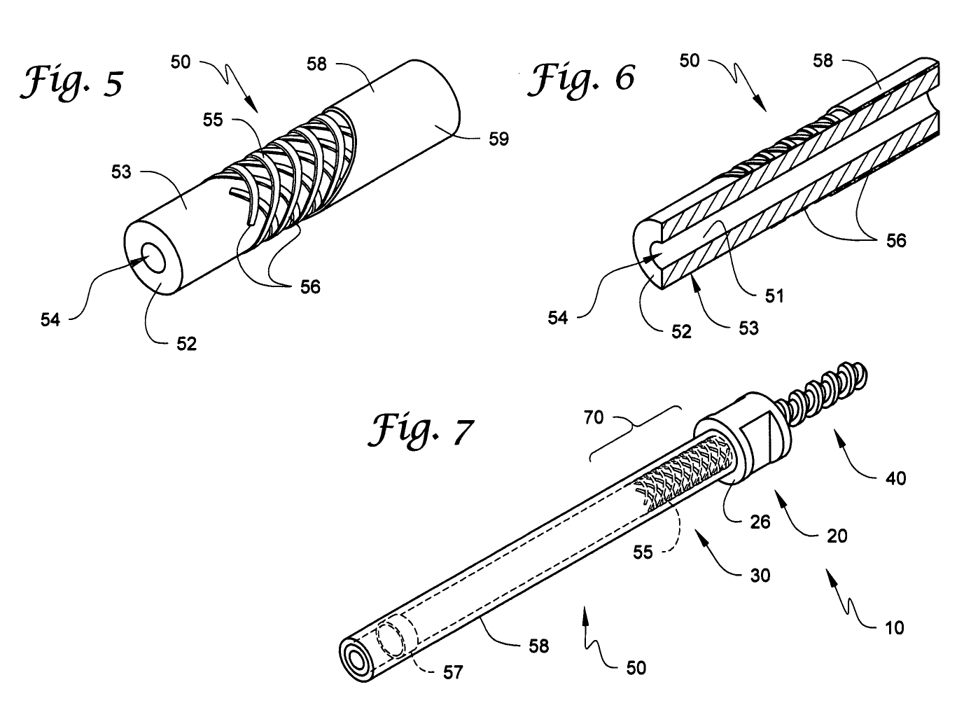

FIGS. 5 & 6 depict views of a portion of one exemplary catheter that may be suitable for use in connection with the threaded connectors (e.g., first connector 30 of FIG. 1) of the present invention. The portion of the catheter 50 that is depicted in FIG. 5 includes an inner body 52, a reinforcing braid 55, and an optional outer jacket 58 that covers the reinforcing braid 55 and the outer surface 53 of the inner body 52.

The inner body 52 of the catheter 50 defines a lumen 54 that extends from the proximal end of the catheter 50 to an infusion section or other portion of the catheter 50 to which fluids are to be delivered using the lumen 54. In many embodiments, the lumen 54 will extend to the distal end or near to the distal end of the catheter 50. The inner body 52 of the catheter 50 can be described as including a wall that extends from an inner surface 51 to an outer surface 53. The lumen 54 is defined by the inner surface 51 of the inner body 52.

The catheter 50 also includes a reinforcing braid 55 located around the outer surface 53 of the inner body 52. The reinforcing braid 55 may include two or more strands 56 that are wound and/or woven around the outer surface 53 of the inner body 52. The spacing between the reinforcing strands 56 may be constant or it may change along the longitudinal length of the catheter 50. The helix angle (i.e., the angle between the strands 56 and a longitudinal axis extending through, e.g., lumen 54) may remain constant or it may change along the longitudinal length of the catheter 50.

The optional outer jacket 58 is attached to the outer surface 53 of the inner body 52 over the reinforcing braid 55. The outer jacket 58 may be provided to present a smoother outer surface 59 for the catheter 50 than could be provided if the reinforcing braid 55 were exposed on the outer surface 53 of the inner body 52.

The reinforcing braid 55 and optional outer jacket 58 may improve the dimensional stability of the catheter 50 both longitudinally as well as radially. For example, the reinforcing braid 55 and optional outer jacket 58 may limit elongation of the catheter 50 along its length (where length is the dimension along which the lumen 54 extends). The reinforcing braid 55 and optional outer jacket 58 may also limit radial expansion of the inner body 52 due to, e.g., the insertion of a connector, fluid pressure within the lumen 54, etc. Other features that may potentially be provided by the reinforcing braid 55 and optional outer jacket 58 may be improved resistance to kinking, crushing, etc. The reinforcing braid 55 and optional outer jacket 58 may also assist in improving the ability of the catheter 50 to transmit torque along its length.

The various components of the catheter 50 may possess a variety of characteristics. For example, the inner body 52 may be an elastically compressible inner body 52, wherein the wall of the inner body 52 is elastically compressible when compressed by, e.g., a connector inserted into the lumen 54 in the inner body 52. The inner body 52 may compress and conform to the shape of a connector inserted into the lumen 54 such that a fluid-tight seal is created between the exterior of the connector and the interior surface 51 of the inner body 52. In one manner, the elastic compressibility of the inner body 52 may be characterized in terms of the durometer of the material used to construct the inner body 52. For example, the inner body 52 may be constructed of material that has a durometer of about 20 Shore A to about 55 Shore D.

As used herein, “elastically compressible” (and variations thereof) means that the wall of the inner body can be compressed from its original uncompressed dimension and, after compression of about 25% (where the distance between the inner surface 51 and the outer surface 53 is about 75% of its original dimension), the thickness of the wall between the inner surface 51 and the outer surface 53 elastically returns to at least about 95% of its original dimension within a time period of about five (5) minutes or less after the compressive force is removed. For example, if a portion of an inner body wall with an original thickness of about 0.100 mm were compressed to a thickness of about 0.075 mm, the compressed portion of the wall would recover to a thickness of about 0.095 mm or more within a period of five (5) minutes or less after the compressive force was removed.

A variety of materials may be used to provide an elastically compressible inner body 52, although the materials selected may preferably be suitable for use in medical devices. Examples may include, e.g., silicone, polyurethane, silicone-urethane thermoplastic copolymer, etc. Also, although the inner body 52 is depicted as being a substantially homogeneous body, it may alternatively be constructed of two or more materials (arranged in layers, as a dispersion, etc.). In some embodiments, the inner body 52 may consist essentially of a medical grade silicone.

Similarly, a variety of materials may be used to construct the reinforcing braid 55. The strands 56 may be inelastic such that they are substantially inextensible under the stresses encountered in normal, expected use of the catheter 50. By “inelastic” (and variations thereof), it is meant that, after elongation of about 10% or more along the length of the strand 56, the strand will recover about 50% or less of the elongation. For example, if an inelastic strand with an original length of 1 centimeter (cm) were stretched to a length of 1.1 cm, the strand would only recover (after removal of the tension force) to a length of 1.05 cm or greater.

Although depicted as monofilaments, each of the strands 56 may alternatively be a composite of two or more filaments. For example, the materials used to construct the strands may include polymers (e.g., a polyester (such as PEN or PET)), metals (e.g., stainless steel, Nitinol, etc.), composite materials, etc. Polymers such as, e.g., polyesters, may provide the desired strength while maintaining a higher level of flexibility than may be possible if, e.g., metallic strands are used. The strands 56 forming the reinforcing braid 55 may be constructed of the same materials or different materials. In one embodiment, the strands 56 may be constructed of PEN or PET (a polyester) and have a cross-sectional dimension (e.g., diameter) of about 0.025 mm to about 0.05 mm. Although the strands 56 used to construct the reinforcing braid may be the same size, they may alternatively be of different sizes.

The materials used for the outer jacket 58 may also potentially be inelastic, although this characteristic is not required. It may, however, be beneficial if the material present on the outer surface 59 of the outer jacket 58 is biocompatible, hydrophobic, and possesses a relatively high tensile strength. One example of a potentially suitable material for the outer jacket 58 is a silicone-urethane thermoplastic copolymer (such as PURSIL 20 or PURSIL 35, available from The Polymer Technology Group, Inc., Berkeley, Calif.). Other potentially useful materials for the outer jacket 58 may include, e.g., polyurethane (e.g., polyurethane 80A or 55D), etc.

Although the outer jacket 58 is depicted as being constructed of a single layer of material, the outer jacket 58 may alternatively be constructed as a composite material of, e.g., two or more layers, a dispersion, etc. For example, an outer jacket 58 may be constructed of a first material that is coated with a second material to, e.g., improve its biocompatibility, reduce surface energy, etc.

In some embodiments, the reinforcing braid 55 may span the entire length of the inner body 52 (i.e., from its proximal end to its distal end). In other embodiments, the reinforcing braid 55 may terminate at a location that is between the distal end of the inner body and the proximal end of the inner body such that a section of inner body 52 located at the distal end of the inner body 52 and extending in the proximal direction is free of the reinforcing braid 55. In some embodiments, the reinforcing braid 55 may be present only over the end portion of the inner body 52 that is occupied by a connector inserted into the lumen 54.

Although the reinforcing braid 55 may terminate short of the distal end of the catheter 50, the outer jacket 58 may extend from the proximal end of the catheter 50 all the way to the distal end of the catheter 50 to protect the outer surface 53 of the inner body 52 over its entire length.

The catheter 50 preferably has a construction in which the outer dimensions (e.g., the diameter in the case of catheter 50 with a circular shape such as that depicted in FIG. 5) remain substantially unchanged when, e.g., a connector is inserted into the lumen 54 of the inner body 52 as compared to the outer dimensions of the catheter 50 when a connector is not located within the lumen 54. Because the reinforcing braid 55 is essentially inextensible and the inner body 52 is elastically compressible, the insertion of a connector into the inner lumen 54 may cause the inner body 52 to conform to the shape of the connector in a manner that forms a fluid-tight seal between the interior surface 51 of the inner body 52 and the outer surface of the connector inserted into the lumen 54. When present, the optional outer jacket 58 may assist in restraining expansion of the inner body 52 in response to the insertion of a connector into the lumen 54.

Although the catheter 50 is depicted as having a generally uniform cross-sectional size along its length, catheter bodies used in connection with the invention may vary in size along their length, e.g., their cross-sectional dimensions may decrease when moving towards the distal ends of the catheter bodies. Likewise, the lumens in catheter bodies of the invention may also have a uniform cross-sectional size over their entire lengths or they may change.

The dimensions of the catheters (and their components) may vary depending on the uses for which they are designed. Although the catheter 50 depicted in FIGS. 5 & 6 has a circular cross-sectional shape, any suitable shape may alternatively be used (e.g., octagonal, elliptical, oval, etc.). For exemplary purposes only, the catheters of the present invention may include inner bodies 52 with an inner diameter (i.e., the diameter of the lumen 54) of about 0.6 mm to about 0.7 mm and outer diameters of about 0.9 mm to about 1.2 mm. The outer jacket 58 may have an outer diameter (which corresponds to the outer diameter of the catheter 50 as a whole) of about 1.4 mm and wall thickness of about 0.1 mm. These dimensions are provided for illustrative purposes only and it should be understood that catheters constructed with dimensions outside of these exemplary ranges may still fall within the scope of the present invention.

FIG. 7 depicts a perspective view of a catheter 50 having the construction depicted and described herein with reference to FIGS. 5-6, where the catheter 50 is attached to a connector body 10 (e.g., the connector body described herein with reference to FIGS. 1-4). Reference to all of FIGS. 1-7 may assist in understanding the connections formed using the catheters and threaded connectors of the invention.

The first connector 30 of the connector body 10 is located within the lumen of a proximal end portion 70 of the delivery catheter 50. As described herein, the inner body 52 of the catheter 50 may compress and conform to the shape of the connector 30, i.e., the threaded outer surface is inserted into the lumen 54 such that a fluid-tight seal is created between the exterior of the first connector 30 and the interior surface 51 of the inner body 52 of the catheter 50. The proximal end of the delivery catheter 50 is depicted as abutting the stop surface 26 of the intermediate section 20 of the connector body 10 (although it may not be required to do so to form a fluid-tight seal).

The delivery catheter 50 may be connected to the first connector 30 of the connector body 10 by first placing the distal tip of the first connector 30 within the lumen 54 at the proximal end of the delivery catheter 50. Then, the connector body 10 and the catheter 50 may be rotated relative to each other (either clockwise or counterclockwise depending on the direction of the threads 34 on the connector 30) such that the connector 30 advances into the lumen 54. The rotation of the connector 30 and the catheter 50 relative to each other and the compression/conformance of the inner body 52 to the connector 30 assist in advancement of the connector 30 into the lumen 54. A clinician may create the force necessary to rotate the connector body 10 and the catheter 50 relative to each other either by hand, or with the assistance of a driven tool. Although relative rotational movement between the connector 30 and the catheter 50 may be beneficial, it may be possible to insert the connector 30 into the lumen (partially or completely) by pure translational motion (i.e., without rotation).

In the depicted catheter 50, the reinforcing braid 55 extends from the proximal end of the catheter 50 (the end closest to the intermediate section of the connector body 10) over at least the proximal end portion 70 in which the connector 30 is located, such that the connector 30 is surrounded by the reinforcing braid 55 when the catheter 50 is attached to the connector 30. Providing the reinforcing braid 55 in the proximal end portion 70 of the catheter 50 may assist in compression and conformance of the inner body 52 to the connector 30 as described herein because the reinforcing braid 55 limits expansion of the inner body 52.

The distal portion of the catheter 50 extending from the distal end of the catheter 50 (the end furthest from the connector body 10) towards the proximal end of the catheter 50 may be free of reinforcing braid 55. Providing a catheter 50 in which the distal portion is free of the reinforcing braid 55 may improve the flexibility of the distal portion of the catheter 50 (as compared to those portions of the catheter 50 in which the reinforcing braid 55 is present). In some embodiments, the reinforcing braid 55 may be limited to the proximal portion of the catheter 50 (where it can assist in restraining the inner body 52 as discussed herein).

The optional outer jacket 58 of the catheter 50 may, however, extend from the proximal end of the catheter 50 all the way to the distal end of the catheter 50. The outer jacket 58 may be provided to, e.g., protect the outer surface 53 of the inner body 52 over its entire length. The outer jacket 58 may also provide some additional structural rigidity to the catheter 50 in those portions where it is present. In some embodiments, the outer jacket 58 may be present over only those portions of the catheter 50 in which the reinforcing braid 55 is present to, e.g., provide a smooth outer surface for the catheter 50.

The catheter 50 depicted in FIG. 7 also includes an optional radio-opaque marker 57 to assist in positioning the delivery catheter 50 at a selected internal body location. Although only one such marker 57 may be provided in connection with catheter 57, more than one marker may be provided to further assist in monitoring the position of the catheter.

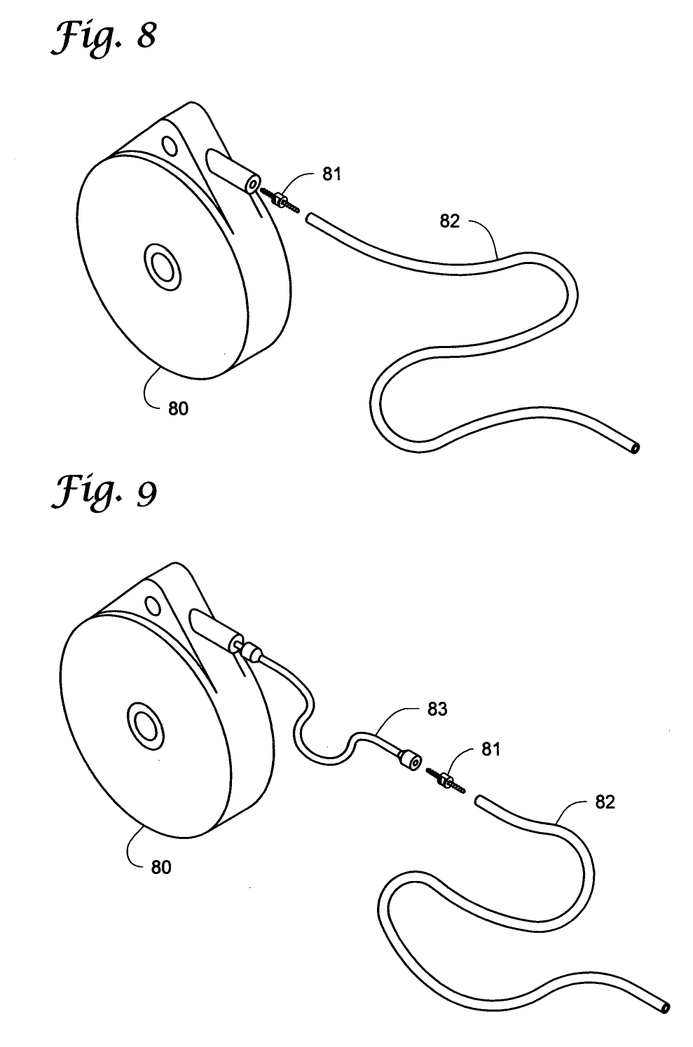

FIG. 8 depicts one illustrative embodiment of an implantable therapeutic substance delivery device 80 that may be used with a threaded catheter connector 81 and a delivery catheter 82 attached to the device 80 using the threaded catheter connector 81. The threaded catheter connector 81 is connected directly to an outlet port on the device 80 and the delivery catheter 82 is attached to the threaded connector 81.

The therapeutic substance delivery device 80 can be used for a wide variety of therapies such as pain, spasticity, cancer, and many other medical conditions. The implantable therapeutic substance delivery device 80 is typically implanted by a clinician in a sterile surgical procedure performed under local, regional, or general anesthesia. Before implanting the therapeutic substance delivery device 80, a delivery catheter 82 is typically implanted with the distal end positioned at the desired therapeutic substance delivery site and the proximal end tunneled to the location where the therapeutic substance delivery device 80 is to be implanted. Then, the therapeutic substance delivery device 80 may be connected to the proximal end of the delivery catheter 82 using the threaded catheter connector 81.

The therapeutic substance delivery device 80 may operate to infuse a therapeutic substance into a patient. Potentially suitable examples of therapeutic substance delivery devices that may be used in connection with the present invention may include, but are not limited to, powered pump assemblies (e.g., piston pumps, diaphragm pumps, peristaltic pumps, etc.) or they may be activated based on pressure to drive fluid out of a reservoir (e.g., using collapsing diaphragms, expanding bladders, etc.). Examples of some potentially suitable therapeutic substance delivery devices may include, e.g., commercially available implantable infusion pumps such as, for example, the SYNCHROMED EL pumps, Models 8626 and 8627, manufactured by Medtronic, Inc., Minneapolis, Minn.

The “therapeutic substance” is a product or substance intended to have a therapeutic effect such as pharmaceutical compositions, genetic materials, biologics, and other substances. Pharmaceutical compositions are chemical formulations intended to have a therapeutic effect such as intrathecal antispasmodics, pain medications, chemotherapeutic agents, and the like. Pharmaceutical compositions are often configured to function in an implanted environment with characteristics such as stability at body temperature to retain therapeutic qualities, concentration to reduce the frequency of replenishment, and the like. Genetic materials are substances intended to have a direct or indirect genetic therapeutic effect such as genetic vectors, genetic regulator elements, genetic structural elements, DNA, and the like. Biologics are substances that are living matter or derived from living matter intended to have a therapeutic effect such as stem cells, platelets, hormones, biologically produced chemicals, and the like. Other substances are substances intended to have a therapeutic effect yet are not easily classified such as saline solution, fluoroscopy agents, and the like.

FIG. 9 depicts an alternative embodiment of an implantable therapeutic substance delivery device 180, a proximal catheter 183, a delivery catheter 182, and a threaded catheter connector 181 according to the present invention. In this embodiment, one end of the proximal catheter 183 is connected to the therapeutic substance delivery device 180. The other end of the proximal catheter may be connected to the delivery catheter 182 using the threaded catheter connector 181. In embodiments where both sides of the connector 181 are threaded, it may be beneficial if the ends of the proximal catheter 183 and the delivery catheter 182 attached to the connector 181 have elastically compressible inner bodies and the other features described herein.

The complete disclosure of the patents, patent documents, and publications cited herein are incorporated by reference in their entirety as if each were individually incorporated.

Illustrative embodiments of this invention are discussed and reference has been made to possible variations within the scope of this invention. These and other variations and modifications in the invention will be apparent to those skilled in the art without departing from the scope of the invention, and it should be understood that this invention is not limited to the illustrative embodiments set forth herein. Accordingly, the invention is to be limited only by the claims provided below and equivalents thereof.

1. A catheter connection system comprising:
a connector body that comprises an intermediate section and a first connector extending from the intermediate section, wherein a bore extends through the first connector and the intermediate section, and wherein the first connector comprises a threaded outer surface; and
a catheter comprising an end portion attached to the first connector, wherein the end portion comprises a lumen that extends through the end portion towards a distal end of the catheter, wherein the lumen is located within an elastically compressible inner body that is surrounded by a reinforcing braid located around an outer surface of the inner body;
wherein, when the first connector is located within the lumen in the end portion of the catheter, the elastically compressible inner body is compressed by and conforms to the shape of the threaded outer surface of the first connector to make a fluid-tight seal between the bore in the connector body and the lumen of the catheter.

2. A system according to claim 1, wherein, when the first connector is located within the lumen in the end portion of the catheter, the outer dimensions of the catheter within the end portion occupied by the first connector remain substantially unchanged as compared to the outer dimensions of the catheter when the first connector is not located within the lumen.

3. A system according to claim 1, wherein the inner body in the end portion of the catheter comprises a wall thickness that is greater than a thread depth of the threaded outer surface of the first connector.

4. A system according to claim 1, wherein the inner body in the end portion of the catheter comprises a wall thickness that is about 25% or more of a diameter of the lumen before the first connector is located therein.

5. A system according to claim 1, wherein the intermediate section of the connector body comprises a pair of wrench flats located on an outer surface of the intermediate section, wherein the wrench flats are located on opposite sides of a longitudinal axis extending along the bore.

6. A system according to claim 1, wherein the reinforcing braid comprises at least one inelastic strand wound around the outer surface of the inner body.

7. A system according to claim 1, wherein the reinforcing braid comprises at least one non-metallic inelastic strand wound around the outer surface of the inner body.

8. A system according to claim 1, wherein the inner body comprises silicone.

9. A system according to claim 1, wherein the inner body consists essentially of silicone.

10. A system according to claim 1, wherein the reinforcing braid and the inner body of the catheter are surrounded by an outer jacket covering the outer surface of the inner body and the reinforcing braid.

11. A system according to claim 1, wherein the system further comprises:
a second connector extending from the intermediate section of the connector body, wherein the bore extending through the first connector and the intermediate body also extends through the second connector; and
a proximal catheter comprising an end portion attached to the second connector, wherein the end portion of the proximal catheter comprises a lumen in fluid communication with the bore.

12. A system according to claim 11, wherein the second connector comprises a threaded outer surface;
wherein the end portion of the proximal catheter comprises an elastically compressible inner body defining the lumen in the proximal catheter, the inner body being surrounded by a reinforcing braid located around an outer surface of the inner body;
and wherein, when the second connector is located within the lumen in the end portion of the proximal catheter, the elastically compressible inner body is compressed by and conforms to the shape of the threaded outer surface of the second connector to make a fluid-tight seal between the bore in the connector body and the lumen of the proximal catheter.

13. A system according to claim 12, wherein, when the second connector is located within the lumen in the end portion of the proximal catheter, the outer dimensions of the proximal catheter within the end portion occupied by the second connector remain substantially unchanged as compared to the outer dimensions of the proximal catheter when the second connector is not located within the lumen.

14. A system according to claim 12, wherein the inner body in the end portion of the proximal catheter comprises a wall thickness that is greater than a thread depth of the threaded outer surface of the second connector.

15. A system according to claim 12, wherein the inner body in the end portion of the proximal catheter comprises a wall thickness that is about 25% or more of a diameter of the lumen before the second connector is located therein.

16. A system according to claim 12, wherein the reinforcing braid comprises at least one inelastic strand wound around the outer surface of the inner body of the proximal catheter.

17. A system according to claim 12, wherein the reinforcing braid comprises at least one non-metallic inelastic strand wound around the outer surface of the inner body of the proximal catheter.

18. A system according to claim 12, wherein the inner body of the proximal catheter comprises silicone.

19. A system according to claim 12, wherein the inner body of the proximal catheter consists essentially of silicone.

20. A system according to claim 12, wherein the reinforcing braid and the inner body of the proximal catheter are surrounded by an outer jacket covering the outer surface of the inner body and the reinforcing braid of the proximal catheter.

21. A method of connecting a catheter to a connector, the method comprising:
providing a connector body that comprises an intermediate section and a first connector extending from the intermediate section, wherein a bore extends through the first connector and the intermediate section, and wherein the first connector comprises a threaded outer surface; and
inserting the first connector in a lumen at an end of a catheter such that the first connector occupies an end portion of the catheter, wherein the lumen is located within an elastically compressible inner body that is surrounded by a reinforcing braid located around an outer surface of the inner body of the catheter;
wherein the inserting comprises rotating the first connector and the catheter relative to each other about an axis extending through the first connector and the lumen, wherein the inner body of the catheter is compressed by and conforms to the threaded outer surface of the first connector to make a fluid-tight seal between the bore in the connector body and the lumen of the catheter.

22. A method according to claim 21, wherein the rotating comprises using a tool on the intermediate section of the connector body to facilitate the relative rotation between the catheter and the first connector.

23. A method according to claim 21, wherein, when the first connector is located within the lumen in the end portion of the catheter, the outer dimensions of the catheter within the end portion occupied by the first connector remain substantially unchanged as compared to the outer dimensions of the catheter when the first connector is not located within the lumen.

24. A method according to claim 21, wherein the inner body in the end portion of the catheter comprises a wall thickness that is greater than a thread depth of the threaded outer surface of the first connector.

25. A method according to claim 21, wherein the inner body in the end portion of the catheter comprises a wall thickness that is about 25% or more of a diameter of the lumen before the first connector is located therein.

26. A therapeutic substance delivery system comprising:
an implantable therapeutic substance delivery device;
a delivery catheter; and
a catheter connector connecting the delivery catheter to the implantable therapeutic substance device, wherein the catheter connector comprises a connector body that comprises an intermediate section and a first connector extending from the intermediate section, wherein a bore extends through the first connector and the intermediate section, and wherein the first connector comprises a threaded outer surface;
wherein an end portion of the delivery catheter is occupied by the first connector, wherein the end portion comprises a lumen that extends through the end portion towards a distal end of the delivery catheter, wherein the lumen is located within an elastically compressible inner body that is surrounded by a reinforcing braid located around an outer surface of the inner body;
and wherein the elastically compressible inner body in the end portion of the delivery catheter is compressed by and conforms to the shape of the threaded outer surface of the first connector to make a fluid-tight seal between the bore in the connector body and the lumen of the delivery catheter.

27. A system according to claim 26, wherein the system further comprises a proximal catheter located between the implantable therapeutic substance delivery device and the catheter connector, wherein the catheter connector further comprises a second connector extending from the intermediate section of the connector body, wherein the bore extending through the first connector and the intermediate body also extends through the second connector;
wherein the proximal catheter comprises an end portion attached to the second connector, wherein the end portion of the proximal catheter comprises a lumen in fluid communication with the bore in the connector body and the implantable therapeutic substance delivery device.

28. A system according to claim 27, wherein the second connector comprises a threaded outer surface;
wherein the end portion of the proximal catheter comprises an elastically compressible inner body defining the lumen in the proximal catheter, the inner body being surrounded by a reinforcing braid located around an outer surface of the inner body;
and wherein the elastically compressible inner body in the end portion of the proximal catheter is compressed by and conforms to the shape of the threaded outer surface of the second connector to make a fluid-tight seal between the bore in the connector body and the lumen of the proximal catheter.
